(19) United States
(12) Patent Application Publication
(10) Pub. No.: US 2024/0023541 A1
(43) Pub. Date: Jan. 25, 2024

(54) APPARATUS AND METHODS FOR CONTROLLING ORGAN TEMPERATURE PRIOR TO AND/OR DURING TRANSPLANTATION PROCEDURES

(71) Applicant: THE UNIVERSITY OF SASKATCHEWAN, Saskatoon (CA)

(72) Inventors: Michael Moser, Corman Park (CA); Jasleen Bhatti, Windsor (CA); Wenjun Zhang, Saskatoon (CA); Anthony Tony, Saskatoon (CA); Bing Zhang, Shanghai (CN)

(21) Appl. No.: 18/029,316

(22) PCT Filed: Sep. 29, 2021
(86) PCT No.: PCT/CA2021/051355

§ 371 Date: Mar. 29, 2023

Related U.S. Application Data
(60) Provisional application No. 63/084,638, filed on Sep. 29, 2020.
(60) Provisional application No. 63/168,958, filed on Mar. 31, 2021.

(51) Int. Cl. A01N 1/02

(52) U.S. Cl. CPC A01N 1/021; A01N 1/0247; A01N 1/0252; A01N 1/0284

(57) ABSTRACT

The invention discloses a temperature control, optionally cooling, apparatus comprising: a first and second and/or plurality of fillable chambers each having a first layer, a second layer, an elongated edge, preferably longitudinal edge, an inlet and an outlet in the elongated, preferably longitudinal edge, and retention members between the first layer and second layer in a series of spaced veins creating a continuous channel extending from the inlet to the outlet; and a method of cooling and or maintaining an organ comprising: a method for cooling, warming and/or maintaining the temperature of an organ prior to or during a transplant procedure, the method comprising: contacting the organ with temperature control, optionally the cooling and/or temperature maintaining, apparatus; securing the apparatus to the organ; attaching the inlets of the first and second and/or plurality of fillable chambers of the apparatus to a circulation fluid source; and/or infusing circulation fluid into the inlet, through the continuous channel, and out the outlet of each of the first and second and/or plurality of fillable chambers of the apparatus.

### RELATED APPLICATIONS

This Patent Cooperation Treaty application claims the benefit of priority of U.S. Provisional Application 63/084,638 filed Sep. 29, 2020 and U.S. Provisional Application 63/168,958 filed Mar. 31, 2021, which are incorporated herein in their entirety.

### FIELD

The disclosure relates to an apparatus that can be used for organ temperature management such as cooling or warming. More specifically, the disclosure relates to an apparatus and methods for regulating the temperature of a donor organ prior to and/or during a transplantation procedure.

### BACKGROUND

Keeping an organ alive as it is transplanted from one person to another is possible because the organ is cooled, flushed of all blood, and filled with special preservation solutions. Of the three steps that are taken, it is possible that the most important is the cooling of the organ to 4° C. which is said to slow the metabolism down approximately 20-fold.

Keeping the kidney at 4° C. is easy enough to accomplish while the kidney is being stored for 4-36 hours by floating the kidney in preservation solution and placing it in two layers of sterile bags that are then placed in ice in a cooler. Unfortunately, at the time of connecting the kidney to its new recipient, in the 30 to 60 minutes that it takes to make the small connections to between the artery of the donor kidney and the new recipient, the kidney warms up to 15° C. or even 25° C. It has been shown recently that the amount of time the kidney has to warm up when it is being sewn in correlates with the survival time of that kidney. A kidney that takes 50-60 minutes to sew in has a 25% higher chance of failure at one year than a kidney that takes 20 minutes. Without a doubt, injury is occurring while the kidney is being sewn in. While a kidney can be kept in a cooler for 24 or even up to 30 hours, when it is taken out of the cold, injury is seen within minutes.

U.S. Pat. No. 6,736,836 (Montgomery) discloses a device and method for maintaining donor organs at reduced temperatures ex vivo, in particular while the organ is being implanted in the recipient, and until reperfusion of the organ, including an organ receiving portion such as, for example, a sleeve or blanket that contacts the organ directly and a temperature controlling mechanism for maintaining the sleeve or blanket at a desired temperature. The sleeve includes a plurality of canals that allow for the circulating of fluid. The sleeve or blanket is secured about the organ by fasteners.

German application DE19962614 (Eichler) appears to disclose a system that includes a cooling sleeve which has coolant fed into a series of fluid channels within the sleeve. There appears to be an entry tube carrying liquid into the sleeve where it appears to travels through all the channels throughout the sleeve, and then out the exit tube of the sleeve. The liquid then appears to travel back to the receptacle where it originated and appears to be recooled by a refrigeration system using an evaporator. There also appears to be a pump that is presumably used to pump the coolant into the sleeve. The patent references using saline solution as the coolant.

PCT application WO1997024088 (Klatz) discloses a method for keeping the organs of a live or dead patient functioning at a reduced metabolic rate while within the body. The invention includes a container/sheet that is wrapped around the whole body, within which there is a network of channels circulating coolant to rapidly cool the body.

Schenkman et al. 1997 describes a technique for cooling a kidney during transplant by supporting the kidney in a slush-filled sterile polyurethane bag. This is bulky and the filling and emptying gets in the way of the sensitive procedure of hooking up the artery and vein and working in a deep hole.

Summers et al. 2011 describe a device for cooling a kidney from 37° C. to 20° C. during minimally-invasive surgery for partial nephrectomies simple bag that is used to cool a kidney by putting the kidney in a fluid filled bag.

Cervantes et al. 2013 discloses a device for laparoscopic transplant surgery and uses an ice slurry.

Longchamp et al. 2019 describes a device made of two rigid silicone sheaths that are permanently attached together creating an adjustable space for the organ and no way of securing the organ in place. It uses ethanol and methylene blue as the circulation fluids.

Keeping the kidney at 4° C. even while it is out of the preservation solution has the potential to greatly decrease the injury to the kidney during that time and may even reduce the risk of rejection (increased injury increases the immune response to a kidney).

A device that can keep the kidney at 4° C. throughout the sewing portion has the potential to help avoid much of the injury that currently occurs to the kidney. Such a device would also allow for more time to make these delicate connections and presumably also more precise connections would be made in an unhurried fashion. Such a device is desirable.

### SUMMARY

The following summary is intended to introduce the reader to various aspects of the disclosure, but not to define or delimit any invention.

A first aspect is a first fillable chamber having a first layer, optionally an upper layer, a second layer, optionally a lower layer, an elongated edge, preferably longitudinal edge, an inlet and an outlet in the elongated edge, preferably longitudinal edge, and retention members between the first layer, optionally the upper layer and the second layer, optionally the lower layer in a series of spaced veins creating a continuous channel extending from the inlet to the outlet, wherein the chamber may be adapted to contact a surface of an organ.

The first fillable chamber can be included in a temperature control apparatus or cooling apparatus that may further comprise a second chamber for contacting a second surface of an organ. A second chamber can confer avoid for example the obstruction of fluid flowing through one chamber that may be folded to adapt to the shape of more than one surface of an organ.

A second aspect of the invention is a temperature control, optionally cooling, apparatus comprising: a first fillable chamber having a first layer, optionally an upper layer, a second layer, optionally a lower layer, an elongated edge, preferably longitudinal edge, an inlet and an outlet in the elongated, preferably longitudinal, edge, and retention members between the first layer, optionally the upper layer and the second layer, optionally the lower layer in a series of spaced veins creating a continuous channel extending from the inlet to the outlet; and a second fillable chamber having a first layer, optionally an upper layer, a second layer, optionally a lower layer, an elongated, preferably longitudinal edge, an inlet and an outlet in the elongated preferably longitudinal edge, retention members between the first layer, optionally the upper layer and the second layer, optionally the lower layer in a series of spaced veins creating a continuous channel extending from the inlet to the outlet, wherein the first chamber and the second chamber may be adapted to contact a surface of an organ.

Another aspect of the invention is a temperature control, optionally cooling, apparatus comprising a plurality of fillable chambers.

Another aspect of the invention includes:
a fillable chamber or a plurality of fillable chambers each fillable chamber comprising
a first layer, optionally an upper layer,
a second layer, optionally a lower layer,
an inlet and an outlet, and
retention members between the first layer, optionally the upper layer and the second layer, optionally the lower layer in a series of spaced veins creating a continuous channel extending from the inlet to the outlet,
optionally wherein each fillable chamber comprises a suspension member, one or more securing members, and/or a vessel holder.

Another aspect of the invention includes a temperature control, optionally cooling, apparatus comprising a plurality of the fillable chambers disclosed herein; wherein the plurality of fillable chambers are adapted to contact a surface of an organ.

The temperature control, optionally cooling, apparatus may also include one or more fastener(s) for example, for securing the first chamber and/or the second chamber to an organ.

One or more of the plurality of fillable chambers comprised in the cooling apparatus may also optionally comprise a suspension member, one or more securing members, and/or a vessel holder.

The temperature control, optionally cooling, apparatus may also include one or more circulation tubings, coupled to or for coupling to the inlet and/or outlet and/or optionally one or more temperature sensors.

The temperature control, optionally cooling, apparatus may be disposable or include one or more components that are disposable.

The first and/or second and/or plurality of fillable chambers may be constructed of a flexible, resilient material. The first and/or second fillable chambers may be constructed of a transparent or translucent material. The first and/or second fillable chambers may also be constructed of other suitable materials including soft plastics, waterproof fabric, and similar materials. The first and/or second fillable chamber may be constructed of vinyl or silicone although other materials can also be used.

As depicted for example in FIG. 5A or 5B first and second fillable chambers 10 can for example be configured such that the first layer, optionally the upper layer 20 and the second layer, optionally the lower layer 30 of each vein 80 of the continuous channel 90 may be in the approximate form of a semi-circle. For example, each extending portion 220 of each vein 80 of the continuous channel 90 can have a cross-sectional distance as measured from the outer surface or inner surface of the first layer, optionally the upper layer 20 and/or outer surface or inner surface of the second layer, optionally the lower layer 30 of about 0.2 cm to about 0.5 cm at the point of greatest thickness. When in use circulation fluid, optionally a cooling fluid or a warming fluid may further expand the cross-sectional distance extending portion 220 of the continuous channel 90 and/or increase, the cross-sectional distance as measured from the outer surface (or inner surface) of the first layer, optionally the upper layer 20 and/or outer surface (or inner surface) of the second layer, optionally the lower layer 30. The width of each spaced vein 80 (e.g. between retention member) may be about 10 mm. The thickness (e.g. height) of each spaced vein 80, measured for example prior to filling with liquid and measured at the point of greatest thickness may be about 2 mm, about 3 mm, about 4 mm or greater and less than for example 5 mm. The width of each vein 80 may be about 10 mm. Each retention member 70 may be about 0.04 mm thick, or about 0.03 mm thick, and about 2 mm wide, or about 1 mm wide, optionally about 1.5 mm wide.

“Lower” layer and “upper” layer are used herein for ease of reference and a person skilled in the art would appreciate that the apparatus may be used such that the lower layer in some embodiments can be the upper layer and vice versa.

The first and/or second fillable chambers and/or plurality of fillable chambers may comprise a plurality of retention members, for example at least 7 retention members. The first and/or second and/or plurality of fillable chambers may comprise at least 8, at least 9 or at least 10 retention members. Each retention member can for example have a thickness of about 0.3 mm.

The inlet and/or outlet may each simply be an opening or may further comprise a fitting. The fitting may be a valve. For example the fitting may be a luer lock attachment.

The inlet and outlet may be located on opposing end portions of the elongated, optionally longitudinal edge of the fillable chamber and/or plurality of fillable chambers.

The inlet and outlet may be positioned so that the inlet may be positioned towards an end portion of each of the first and/or second and/or plurality of fillable chambers and/or the outlet may be positioned in a middle portion of each of the first and/or second and/or plurality of fillable chambers.

The elongated edge connects the first and second layer of a fillable chamber and comprises the inlet and/or outlet. The elongated edge, may be convex or concave or angled for example to form approximately a V-shape, as in for example a hexagon as shown in FIG. 10A or may in preferable embodiments be a longitudinal edge. A longitudinal edge can for example be present when the fillable chamber and/or plurality of fillable chambers, is/are substantially quadrilateral, for example substantially a parallelogram (e.g. square, rectangle, or rhombus), substantially trapezoidal, or other shapes for example substantially triangular or a semi-circle. When the elongated edge is convex as in an oval shaped or circular shaped fillable chamber or is concave, the inlet and outlet may be separated by a distance. For example, in the case of a circular or oval shaped fillable chamber, the inlet may be about opposite the outlet for example on opposite sides of the centre. In a concave shaped fillable chamber and/or plurality of fillable chambers, the inlet and outlet may be located towards each end of the concave edge.

As described herein, the continuous channel may be configured in 5 various patterns that permit continuous flow, such as a solenoid pattern. The continuous channel may also be configured in a U-shape pattern.

The inlet and/or outlet fittings may be releasably attached to the circulation tubing. The circulation tubing can be used to flow a circulation fluid, optionally a cooling fluid or a warming fluid through the continuous channel of the chamber(s).

In other examples, the first and/or second and/or plurality of fillable chambers may have markings for example to indicate orientation in use, for example the markings may identify the portion that covers or is proximal to for example the ureter. Alternatively, the markings may identify which portion of the fillable chambers and/or plurality of fillable chambers should be oriented generally towards the patient's head and/or the portion that should be oriented generally towards the patient's feet. Markings may also be used to indicate inlet and/or outlet and/or markings to indicate where to place an organ, and/or markings to indicate where the fasteners should be fastened.

The apparatus or kit can be for use with any organ, including appendages or tissue etc. to be transplanted or reattached. The organ may be a kidney, a liver, a pancreas, a heart or a hand or face or any transplant organ or tissue flap. It is also understood that transplant as used herein can include re-attachment.

The apparatus or kit can comprise a plurality of fillable chambers of the same or various shapes, for example 2 fillable chambers, 3 fillable chambers, 4 fillable chambers, and/or 5 fillable chambers or more, for example 10, 20, 30, 40 or 50 or any number between 2 and 100. The chambers can be adapted to be modular and used in various combinations according to the dimensions and configuration of an organ to be cooled, warmed and/or maintained at a specific temperature and/or range of temperatures.

For example, the apparatus or kit may be for a heart and may for example comprise a plurality of fillable chambers, optionally a first, second and third fillable chamber, for example shaped and/or sized to provide suitable cooling and/or warming and/or temperature maintenance contact and suitable access to the aorta, pulmonary artery and vena cava as well as one or more chambers such as the left or right atrium. One or more of the first, second or third fillable chambers may be for example trapezoidal in shape, or be of different sizes, for example the first fillable chamber may be larger than the second chamber which may be equal to or larger than the third chamber, wherein the first, second and third fillable chambers are adapted to contact a surface or portion of the heart. In a further example, the fillable chambers are connected using for example a fastener.

In another example, the apparatus or kit may be for a liver and may for example comprise a plurality of fillable chambers, optionally a first and second fillable chamber, wherein one or more of the first and second fillable chambers are of different sizes or shapes for example shaped and/or sized to provide suitable cooling and/or warming and/or temperature maintenance contact with one or more portions of the liver. For example one or more may be trapezoidal in shape, or wherein the first fillable chamber may be larger than the second chamber. In an alternate example, the apparatus may be for a liver and may for example comprise a first, second, third, and fourth fillable chamber, wherein one or more of the first, second, third, and fourth fillable chambers may be for example shaped and/or sized to provide suitable cooling and/or warming and/or temperature maintenance contact with one or more portions of the liver, for example triangular in shape, optionally wherein the first and second fillable chambers may for example be larger than the third and fourth fillable chambers, wherein the first, second, third, and fourth fillable chambers may for example be adapted to contact a surface of a liver, optionally a particular lobe. In a further example at least two of the fillable chambers are connected using for example a fastener.

The fillable chambers can be used as modular components depending on an organ to be transplanted and/or its size. For example, larger and smaller fillable chambers or fillable chambers of different shapes can be connected to form laterally connected apparatus (e.g. where the chambers are connected along their lateral edges via clips, buttons, clasps and the like to provide cooling to a plurality of different shaped and sized organs.

In another example, the apparatus or kit may be for a lung and may for example comprise a first and second fillable chamber and optionally a third and fourth fillable chamber particularly in the case of a double lung transplant, wherein the first and second fillable chambers may be for example tapered with a narrower end and a wider end approximating the shape of a lung. Optionally the chambers may be used in a modular fashion where a first and second fillable chamber are assembled with a third and fourth fillable chamber, wherein the first and second fillable chambers may be larger than the third and fourth fillable chambers, and wherein the first and second fillable chambers may be adapted to contact a surface of a lung such as a lower lung portion which tends to be larger (e.g. wider) than an upper lung portion and the third and fourth fillable chambers may be adapted to contact the surface of a lung such as an upper lung portion which tends to be tapered relative to the lower lung portion. In a further example at least two of the fillable chambers are connected using for example a fastener.

In another example, the apparatus or kit may be for a pancreas and may for example comprise a first, second, third, and fourth fillable chamber, wherein one or more of the first and second fillable chambers may be for example oblong in shape and the third and fourth fillable chambers may be for example circular in shape, wherein the first, second, third, and fourth fillable chambers may for example be adapted to contact the surface of a pancreas. In a further example at least two of the fillable chambers are connected using for example a fastener.

The apparatus or kit can be used for cooling an organ or warming an organ.

Another aspect of the invention is a method for cooling, warming and/or maintaining the temperature of an organ prior to and/or during a transplant procedure, the method comprising: contacting the organ with a temperature control, optionally cooling, apparatus described herein; securing the apparatus to the organ; attaching the inlets of the first and second fillable chambers (or plurality) of the apparatus to a circulation fluid source; and infusing circulation fluid into the inlet, through the continuous channel, and out the outlets of each of the plurality of fillable chambers, optionally the first and second fillable chambers of the apparatus prior to and/or during transplantation. Circulation fluid and circulation fluid source is understood to also include fluid and a source where such fluid is for example less than body temperature and which can be used to cool or warm the organ and/or maintain a desired temperature. The method can comprise initially cooling, followed by warming or repeated warming and cooling steps. A person skilled in the art would recognize that the order of one or more of the method steps may be altered. For example, it is possible to attach the inlet and/or outlet to a circulation fluid source before securing the apparatus to the organ.

Warming fluid can be used where warming (or rewarming) an organ is desired and can be used, instead of, or subsequent to cooling fluid. Cooling fluid is understood to be a circulation fluid that can flow through the continuous channel and can for example be used to lower the surface temperature of an organ to a target or desired temperature as compared to for example the organ surface temperature prior to the step of infusion of the cooling fluid, and/or for example to maintain the temperature of an organ at a target or desired temperature or temperature range that is for example the same as or lower than, the temperature or temperature range of the organ prior to the infusion of the cooling fluid. It may also be used to limit the temperature increase which would still be understood as cooling. For example an organ may be transported in a cooling box and the apparatus described herein may be used to maintain a desired temperature once the organ is removed from the cooling box and exposed to the operative field. Warming fluid is understood to be a circulation fluid that can flow through the continuous channel and can for example be used to increase the surface temperature of an organ to a target or desired temperature as compared to for example the organ surface temperature prior to the step of infusion of the warming fluid and/or for example to maintain the temperature of an organ at a target or desired temperature or temperature range that is for example the same as, or higher than, the temperature or temperature range of the organ prior to the infusion of the warming fluid. For example, it may be desirable to maintain an organ above room temperature. The warming fluid may used to limit the temperature decrease which would still be understood as warming.

The contacting can comprise placing the first fillable chamber on a surface of the organ, placing the second fillable chamber on another surface of the organ.

The first fillable chamber may be placed and/or may be sized and/or shaped or used in combination with other fillable chambers to contact a substantial portion of an organ to be cooled (and/or optionally warmed). A substantial portion of the organ can be for example at least about 50% of a first surface of the organ, or at least about 60% of a first surface of the organ, or at least about 70% of a first surface of the organ, or at least about 80% of a surface of the organ. In some embodiments, the first fillable chamber may be placed and/or may be sized and/or shaped or used in combination with other fillable chambers to contact at least about 90%, of a surface of the organ.

Similarly, the second fillable chamber and/or a plurality of chambers may be placed and/or may be sized and/or shaped to contact a substantial portion of an organ to be cooled. A substantial portion of the organ can be for example at least about 50% of a first surface of the organ, or at least about 60% of a first surface of the organ, or at least about 70% of another surface of the organ, or at least about 80% of another surface of the organ. In some embodiments, the second fillable chamber may be placed and/or may be sized and/or shaped to contact at least about 90% of a second surface of the organ.

The step of securing the apparatus to the organ can for example comprise fastening one or more fastener(s) for securing the first and/or second fillable chambers or a plurality of fillable chambers to the organ and/or appendage.

The method may further include connecting the inlets of each of the first and/or second fillable chambers and/or plurality of fillable chambers to a fitting. The fitting may be a valve. The fitting may be a luer lock attachment.

The method may include attaching circulation tubing to the circulation fluid source. The circulation fluid source may be a circulation fluid receptacle comprising a circulation fluid. The circulation fluid may for example be warm or cold saline, warm or cold water, or ice water. Other circulation fluid can also be used. The method can also be for warming an organ and the circulation fluid source in such case would warm for example warm saline or warm water. Preferably, the circulation fluid may be sterile. The receptacle may be a bag with a sealable opening for receiving circulation fluid and an aperture for dispensing the circulation fluid, via for example the circulation tubing.

The infusing step can be performed to provide a desired flowrate. For example, the infusing step can be performed to achieve a flowrate of between about 600 cc/hour to about 1000 cc/hour. The method may include the infusing being suitable to achieve a flowrate of about 750 cc/hour.

The desired flowrate can optionally be achieved using a pump.

The desired flowrate can also be achieved using gravity.

The infusing step can be performed to maintain the organ surface temperature at a target or desired temperature or to reduce the organ surface temperature to any temperature.

The infusing step can be performed to maintain or reduce the organ surface temperature to between about 3° C. and about 6° C. The method may include the infusing being suitable to maintain or reduce the organ surface temperature to between about 3.4° C. and about 4.6° C.

The infusing step can be performed to maintain organ surface temperature at about 3° C., at about 4° C., at about 5° C., or at about 6° C. (e.g. the target temperature). The infusing step can be performed to maintain the organ surface temperature at about 3-4° C. The infusing step can be performed to maintain the organ surface temperature at about 4-6° C. Other temperatures or ranges include about 8° C. or about 3-10° C. or about 16° C. to about 25° C.

As desired temperature can be more readily controlled, and with minimal interference, the apparatus may allow for increased time to perform an anastomosis. For example, the apparatus may extend anastomotic time without increasing ischemia time, for example, it may increase the time available for safely performing the anastomosis to up to 2 hours or up to 3 hours.

If the method is or comprises a step of warming the organ, the infusing step can be performed to increase the organ surface temperature, typically gradually, to about room temperature or more typically about body temperature, optionally between about 35° C. and about 38.5° C. The infusing step can also be performed to increase the organ surface temperature to and/or maintain the organ surface temperature between about 4° C. and about 39° C., optionally at about 7° C., at about 8° C., at about 9° C., at about 10° C., at about 15° C., at about 20° C., at about 25° C., at about 30° C., at about 35° C., at about 36° C., at about 37° C., at about 38° C., or at about 39° C., preferably, at about room temperature or at about body temperature, optionally between about 35° C. and about 38.5° C.

The infusion step can also be performed to increase the organ surface temperature to and/or maintain the organ surface temperature between about 10° C. and about 38° C., between about 15° C. and about 38° C., optionally between about 16° C. to about 25° C., between about 20° C. and about 38° C., between about 25° C. and about 38° C., or between about 30° C. and about 38° C. The infusion step can also be performed to increase the organ surface temperature to between about 10° C. and about 38° C., between about 15° C. and about 38° C., between about 20° C. and about 38° C., between about 25° C. and about 38° C., or between about 30° C. and about 38° C.

The infusion step can also be performed to increase and/or decrease the organ surface temperature gradually.

The infusing step can be performed to keep or reduce the organ surface temperature to a temperature of hypothermic metabolic arrest of the organ.

The infusion may be performed for a period of time, for example at least or about 40 minutes, or between 5 minutes and 3 hours, optionally the length of time necessary for the completion of the procedure.

Another aspect of the invention may be a method of making the temperature control, optionally cooling, apparatus comprising contacting a first layer and a second layer of flexible, resilient material with a heating device to fuse areas thereby inserting retention members of the first layer and the second layer, thereby creating the series of spaced veins that together form the continuous channel. In such embodiments, the edges of the first layer and the second layer are also sealed or otherwise joined, with openings for an inlet and an outlet.

The continuous channel may be configured in a solenoid pattern. The continuous channel may be configured in a U-shape pattern.

The resilient material may be a vinyl or silicone.

The heating device may be a sealer.

Another aspect of the invention may include a kit comprising one or more components of the temperature control, optionally cooling, apparatus.

The kit may include one or more fastener(s) and/or side fasteners. The kit may include a pump. The kit may include a circulation tubing. The kit may include a circulation fluid receptacle. One or more components of the kit may be packaged in sterile condition.

Another aspect of the invention may include use of the methods described herein and/or the kits described herein for cooling and/or warming and/or maintaining the temperature of an organ prior to and/or during a transplant procedure.

### DRAWINGS

The drawings included herewith are for illustrating various examples of articles, methods, and apparatuses of the present specification and are not intended to limit the scope of what is taught in any way. In the drawings:

FIG. 1A is an image depicting the top or bottom view of an example temperature control, optionally cooling, apparatus, wherein the apparatus is contacting an organ. FIG. 1B is an image depicting the left lateral-side view (which is left open, but may be closed as in FIG. 1C, using for example a Velcro attachment, to allow for greater contact of the cooling device with the organ) of the temperature control, optionally cooling, apparatus of FIG. 1A. FIG. 1C is an image depicting the right lateral-side view, which is closed with tape in this embodiment, but in other embodiments may be closed with for example a Velcro attachment, to allow for greater contact of the cooling device with the organ) of the temperature control, optionally cooling, apparatus of FIG. 1A.

FIG. 2 is an image depicting the perspective view of the exterior of the fillable chamber of the temperature control, optionally cooling, apparatus in FIG. 2A.

FIG. 3A. is an image depicting the top or bottom view of the exterior of an example of a fillable chamber of the temperature control, optionally cooling, apparatus in FIG. 1A, FIG. 1B, and FIG. 1C. FIG. 3B is a schematic drawing of the top or bottom view of the interior of the fillable chamber of FIG. 2, wherein the continuous channel is configured in a solenoid pattern having 7 retention members. FIG. 3C is a schematic drawing of the top or bottom view of the interior of the fillable chamber of FIG. 2, wherein the continuous channel is configured in a solenoid pattern having 9 retention members.

FIG. 4 is a schematic drawing of the top or bottom view of the interior of an example of a fillable chamber of an example temperature control, optionally cooling, apparatus, wherein the continuous channel is configured in a U-shape pattern.

FIG. 5A is a schematic drawing of the side view of an elongated edge of the exterior of the fillable chamber of FIG. 2, FIG. 3A, and FIG. 3B having 7 retention members. FIG. 5B is a schematic drawing of the right-side or left-side view of the exterior of the fillable chamber of FIG. 3C having 9 retention members

FIG. 6A is an image of the top or bottom view of an example of a circulation tubing. FIG. 6B is an image of the front or back view of an example of a circulation fluid receptacle, wherein the circulation fluid receptacle is a bag.

FIGS. 7A and 7B are schematics drawings of a temperature control, optionally cooling, apparatus according to an embodiment of the present invention.

FIGS. 8A and 8B are schematic drawings of a temperature control, optionally cooling, apparatus according to an embodiment of the present invention. FIG. 8A is schematic of a top view of the temperature control, optionally cooling, apparatus. FIG. 8B is a schematic of a perspective view of the temperature control, optionally cooling apparatus FIG. 8A.

FIG. 9A, 9B, 9C, and 9D are images of various views of an example of a temperature control, optionally cooling, apparatus shown in use with an organ, according to an embodiment of the present invention.

FIGS. 10A and 10B are schematic drawings of a fillable chamber according to an embodiment of the present invention.

### DETAILED DESCRIPTION

Various apparatuses or processes will be described below to provide an example of an embodiment of the claimed subject matter. No embodiment described below limits any claim and any claim may cover processes or apparatuses that differ from those de-scribed below. The claims are not limited to apparatuses or processes having all of the features of any one apparatus or process described below or to features common to multiple or all of the apparatuses described below. It is possible that an apparatus or process described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular.

As used in this specification and the appended claims, the singular forms “a”, “an” and “the” include plural references unless the content clearly dictates otherwise. It should also be noted that the term “or” is generally employed in its sense including “and/or” unless the content clearly dictates otherwise.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

In understanding the scope of the present disclosure, the term “comprising” and its derivatives, (such as “comprise” and “comprises”), “having” (and any form of having, such as “have” and “has”), “including” (and any form of including, such as “include” and “includes”) or “containing” (and any form of containing, such as “contain” and “contains”), as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”. For ranges described herein, subranges are also contemplated, for example every, 0.1 increment there between. For example, if the range is 10° C. to about 38° C., also contemplated are for example 10.1° C. to about 38° C., 10° C. to about 37.9° C., 10.1° C. to about 37.9% and the like.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Disclosed herein is a temperature control, optionally cooling, apparatus. As used herein, the term “organ” refers to a range of organs, including organs of the human chest and/or abdominal regions, such as for use in connection with the transplant of a kidney, liver, lung, heart or pancreas or small bowel. The temperature control, optionally cooling, apparatus of the invention also will be useful for the transplant of other organs such as appendages e.g. human hand, foot, or finger, and/or a face and/or uterus. As used herein, the term appendages includes for example hands, feet, fingers, toes, or free flap (for example as in reconstructive plastic surgery—a muscle or flap of skin, fat, and muscle, or even bone that is served by a single artery or vein can be ‘transplanted’ on the same person to another part of the body).

As will be described in detail below, the temperature control, optionally cooling, apparatus can be used to cool an organ prior to and/or during a transplant procedure. As would be understood by a person skilled in the art, the apparatus can also be used to warm an organ, for example to gradually rewarm the organ to body temperature after the period of cold ischemia. The rewarming may be gradual, for example using solutions at progressively warmer temperatures and/or one or more sensors may be involved to control the rate of warming.

In some examples, the temperature control, optionally cooling, apparatus or one or more parts thereof may be disposable. In other examples, the temperature control, optionally cooling, apparatus or one or more components thereof may be reusable. Any component that can be sterilized can be reusable. For example, one or more of the fillable chambers, temperature sensor(s), tubings and the like may be disposable. Pumps, circulation fluid receptacle and/or valves (feedback valve) are typically reusable.

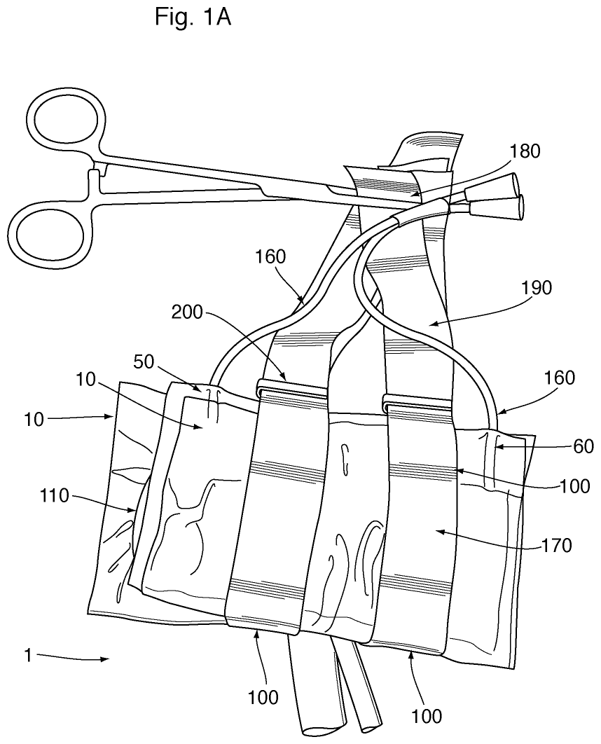

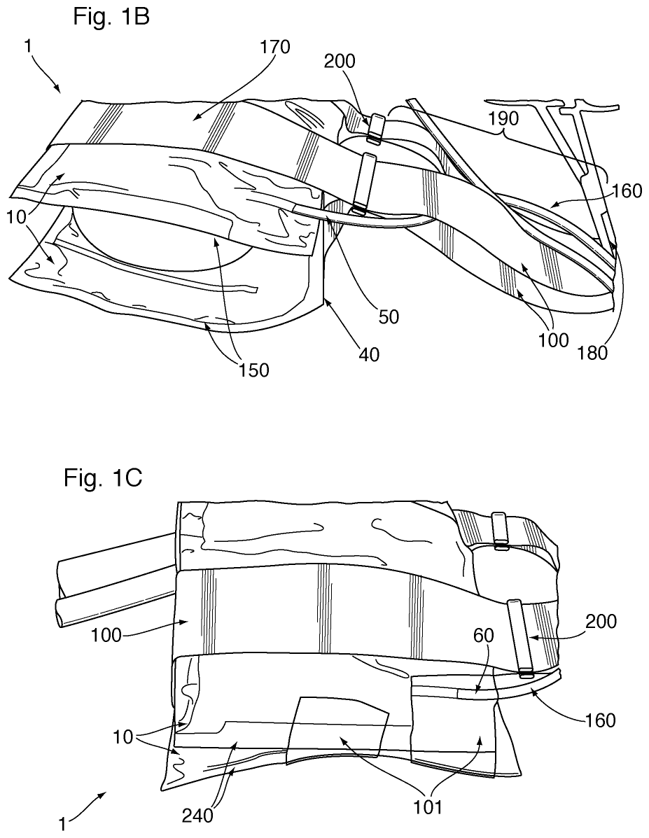

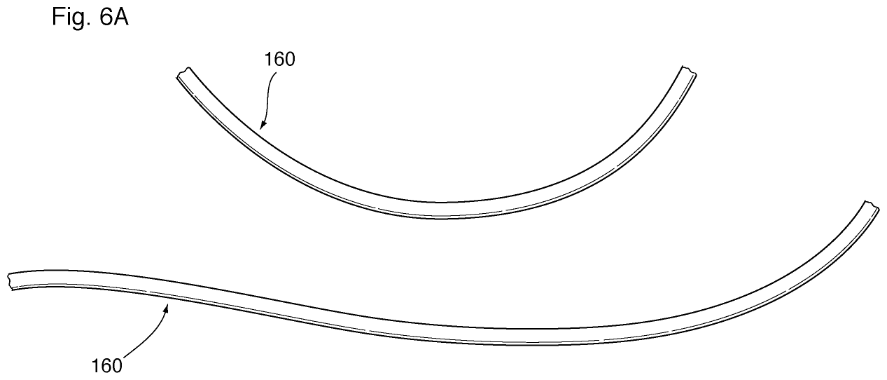

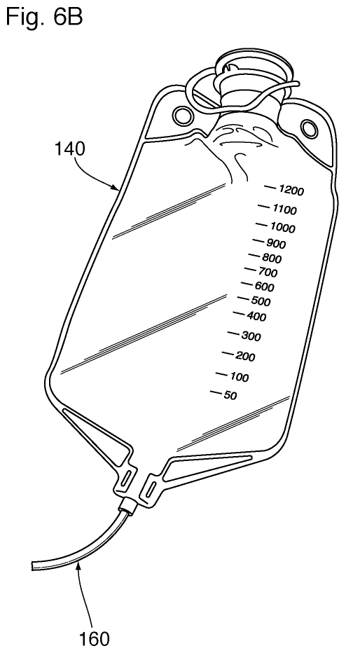

Referring first to FIG. 1A, 1B, and 1C, in the examples shown, the temperature control, optionally cooling, apparatus 1 includes a first fillable chamber 10 (shown in FIGS. 2-5) having an first layer, optionally an upper layer 20, a second layer, optionally a lower layer 30, a longitudinal edge 40, an inlet 50 and an outlet 60 in the longitudinal edge 40, and retention members 70 between the first layer, optionally the upper layer 20 and the second layer, optionally the lower layer 30 in a series of spaced veins 80 creating a continuous channel 90 extending from the inlet 50 to the outlet 60; a second fillable chamber 10 having a first layer, optionally an upper layer 20, a second layer, optionally a lower layer 30, a longitudinal edge 40, an inlet 50 and an outlet 60 in the longitudinal edge 40, retention members 70 between the first layer, optionally the upper layer 20 and the second layer, optionally the lower layer 30 in a series of spaced veins 80 creating a continuous channel 90 extending from the inlet 50 to the outlet 60; fasteners 100 for securing the first chamber 10 and the second chamber 10, wherein the first chamber 10 and the second chamber 10 are adapted to each contact a surface of an organ 110; and circulation tubing 160 (also shown in FIG. 6A). In alternative examples, the temperature control, optionally cooling, apparatus 1 does not include one or more fasteners 100. In alternative examples, the temperature control, optionally cooling, apparatus 1 does not include circulation tubing 160. In alternative examples, the temperature control, optionally cooling, apparatus 1 comprises at least one fillable chamber 10. In other examples, the temperature control, optionally cooling, apparatus 1 comprises a plurality of fillable chambers.

In the examples shown, the first fillable chamber 10 is depicted as contacting the first surface, optionally an upper surface of an organ 110 and the second fillable chamber 10 is depicted as contacting the second surface, optionally the lower surface of the organ 110.

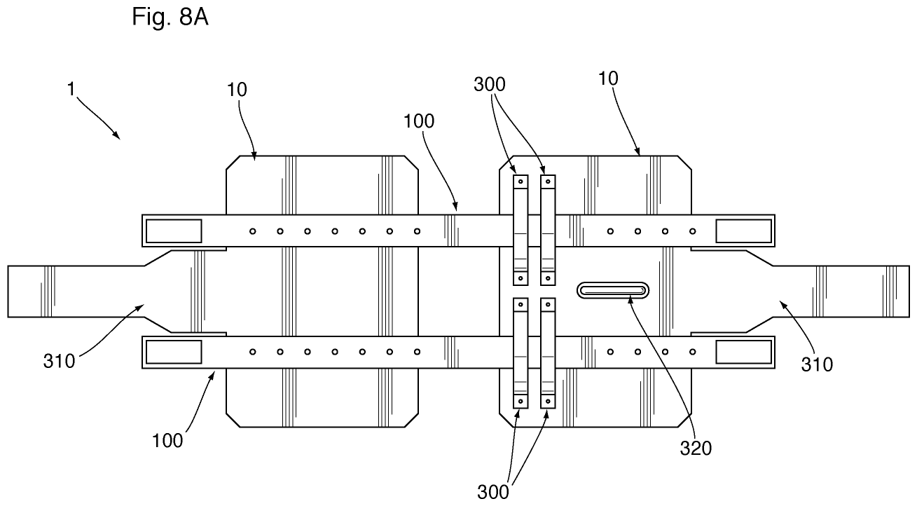

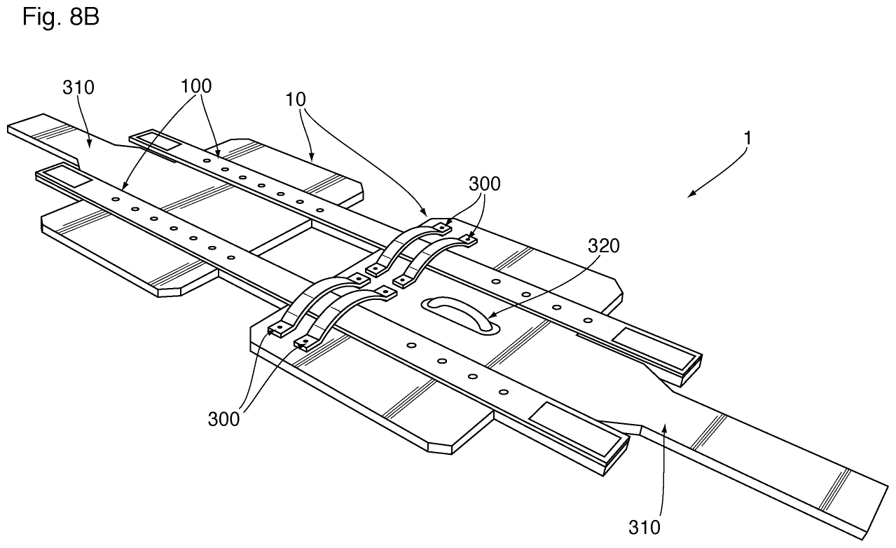

The fasteners 100 can be adjustable (e.g. FIG. 1A) or fixed (e.g. FIG. 8A).

In the examples shown, two fasteners 100 which are adjustable are depicted as securing the first chamber 10 and the second chamber 10, wherein the first chamber 10 and the second chamber 10 are adapted to each contact a surface of an organ 110. Specifically, the fasteners 100 are wrapped around both the first fillable chamber 10 and the second fillable chamber 10 and extend and meet at a point further than the first and second fillable chambers 10, comprising a chamber portion 170, and a first portion, optionally an upper portion 180 of an extended portion 190 of the fasteners 100. The fasteners 100 are secured together using for example clips 200. The extended portion 190 is optional and when present the first portion, optionally the upper portion 180 of the extended portion 190 can be engaged to suspend the organ, for example by means of a clamp such as a Kelly clamp as shown in FIG. 1A. The chamber portion 170 of the fastener 100 refers to the portion extending from the longitudinal edge 40 of the first chamber 10 to the longitudinal edge 40 of the second chamber 10. In the example shown, the fasteners 100 wrapped around both the first and second fillable chambers 10 and extending beyond the chambers can be non-adhesive straps. Additionally, in the examples shown in FIGS. 1A and 1C, side fasteners 101, can be used to connect the first lateral edge 240 of each of the fillable first and second fillable chambers 10, and in this example, the side fasteners 101 are Steri-Strips™. In alternative examples, side fasteners 101 are used to connect the second lateral edge 150 of each of the fillable first and second fillable chambers 10. In some examples, side fasteners 101 can be adjustable or fixed.

In alternative examples, the fasteners 100 and/or side fasteners 101 may be adhesive straps, optionally tape. In alternative examples, the fasteners 100 and/or side fasteners 101 may be Velcro™ or Steri-Strips™, or other hook and loop fasteners. In alternative examples the fasteners 100 and/or side fasteners 101 may be snaps, buttons, buckles or similar fasteners that keep an organ wrapped securely and allow the temperature control, optionally cooling, apparatus 1 to be removed. In alternative examples, the fasteners 100 and/or side fasteners 101 do not extend past the first and/or second fillable chambers 10. In alternative examples, one or more of the fasteners 100 and/or side fasteners 101 may be attached to one of the first and/or second fillable chambers 10. In alternative examples, the one or more fasteners 100 and/or side fasteners 101 are attached to both the first and second fillable chambers 10 and to allow the first and/or second fillable chambers 10 to each contact a surface of the organ 110. In alternative examples, one or more fasteners 100 and/or side fasteners 101 is/are attached to each of the first and/or second fillable chambers 10. In alternative examples, no fasteners 100 and/or side fasteners 101 are attached to the first and/or second fillable chambers 10. In alternative examples, one or more fasteners 100 and/or side fasteners 101 are only wrapped around the first and second fillable chambers 10, with no side fasteners 101 connected the first lateral edge 240 or second lateral edge 150 of the first and second fillable chambers 10. In alternative examples, side fasteners 101 are only used on the first lateral edge 240 and/or the second lateral edge 150 of the first and second fillable chambers 10. In alternative examples, only side fasteners 101 are used on the first lateral edge 240 and/or the second lateral edge 150 of the first and second fillable chambers 10 and no fasteners 100 are used.

In the examples shown, the inlet 50 of the first fillable chamber 10 may be configured to be aligned with the outlet 60 of the second fillable chamber 10 and the inlet 50 of the second fillable chamber 10 may be configured to be aligned with the outlet 60 of the first fillable chamber 10, optionally when in use. In alternative examples, the inlet 50 of the first fillable chamber 10 may be configured to be aligned with the inlet 50 of the second fillable chamber 10 and the inlet 50 of the second fillable chamber 10 may be configured to be aligned with the inlet 50 of the first fillable chamber 10, optionally when in use.

In the examples shown, a circulation tubing 160 may be releasably connected to the inlets 50 of each of the first and/or second fillable chambers 10. Specifically, the circulation tubing 160 may be placed in the inlets 50 of each the first and/or second fillable chambers. In alternative examples, the circulation tubing 160 may be releasably or permanently connected to the inlets 50 and/or the outlets 60 of each of the first and/or second fillable chambers 10. In alternative examples, the circulation tubing 160 may be connected to a fitting in the inlet 50 or outlet 60 of a fillable chamber 10. In alternative examples, the fitting may be a valve. In alternative examples, the fitting may be a luer lock attachment. In other examples, the fitting may be any valve that allows the removal of air bubbles. In another example, the fitting may be a three-way valve. In some examples, the fitting may be a three-way stopcock.

In alternative examples, there may be a kit including one or more fillable chambers 10. In alternative examples, the kit may include one or more fasteners 100 and/or one or more side fasteners 101. In alternative examples, the kit may include one or more circulation tubing 160. In alternative examples, the kit may include one or more circulation fluid receptacles 140. In alternative examples, the kit may include a disposable or reusable temperature sensor 250. In alternative examples, the kit may include a transmitting wire 260. In alternative examples, the kit may include any thermostatic control. In alternative examples, the kit may include a valve, optionally a three-way valve. In alternative examples, the kit may include a discard bag 290 and/or circulation tubing 160. In other examples, the kit may include a pump, optionally battery operated, optionally attached to a receiver 270. In other examples, the kit may include a flow valve, optionally attached to a receiver 270. In alternative examples, the kit may include one or more vessel holders 320. In some examples, the one or more vessel holders 320 are attached the one or more fillable chambers 10. In alternative examples, the one or more vessel holders 320 are not attached to the one or more fillable chambers 10. In alternative examples, the kit may include one or more securing members 300. In alternative examples, the kit may include one or more suspension members 310. In alternative examples, the kit may include one or more adhesive, snap, clip or other for connecting different components included in the kit. In alternative examples, the components included in the kit may be packaged in sterile condition.

Referring now to FIG. 2A, in the example shown, the fillable chamber 10, has a first layer, optionally an upper layer 20 that may be above the second layer, optionally the lower layer 30. In alternative examples, the first layer, optionally the upper layer 20 may be beneath the second layer, optionally the lower layer 30. For example, either or both outside surfaces of the layers can be configured to contact an organ when in use.

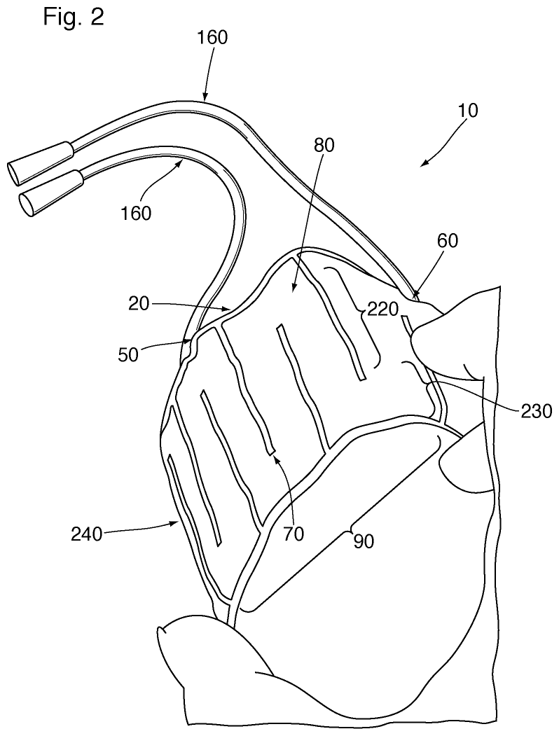

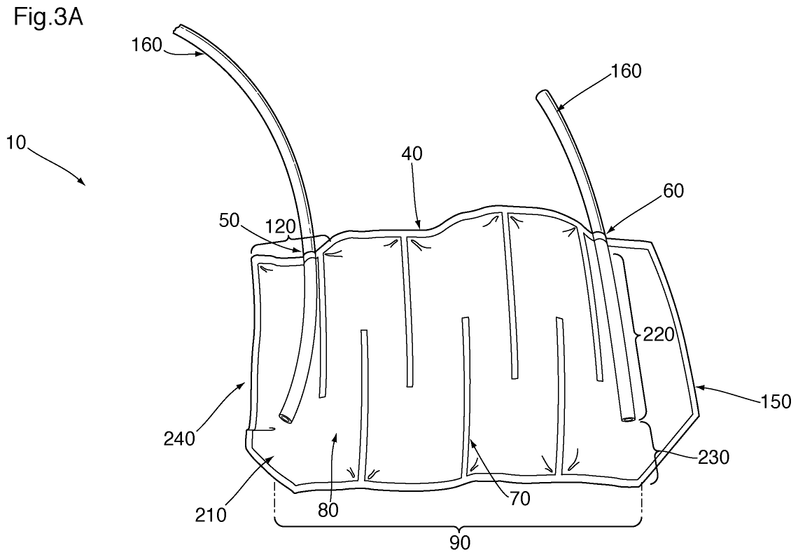

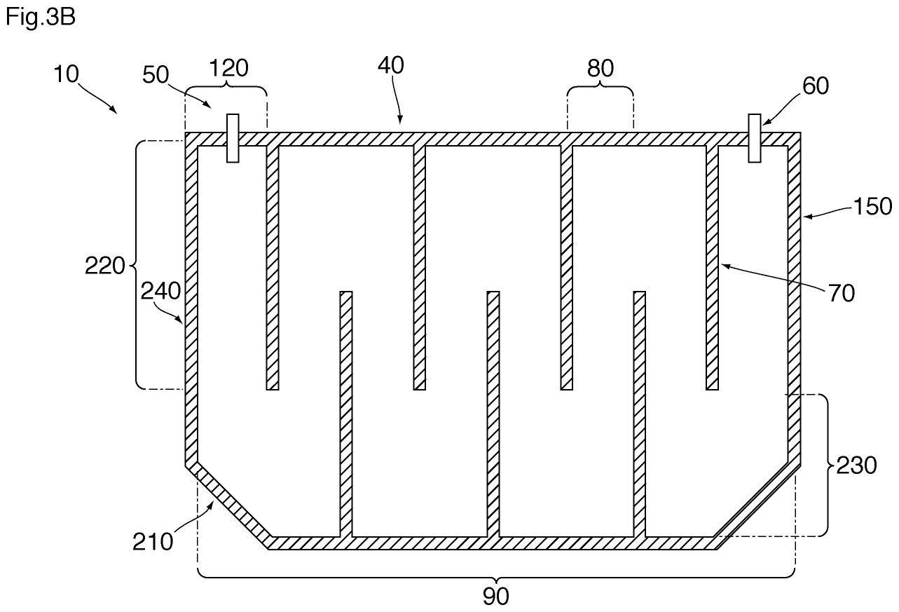

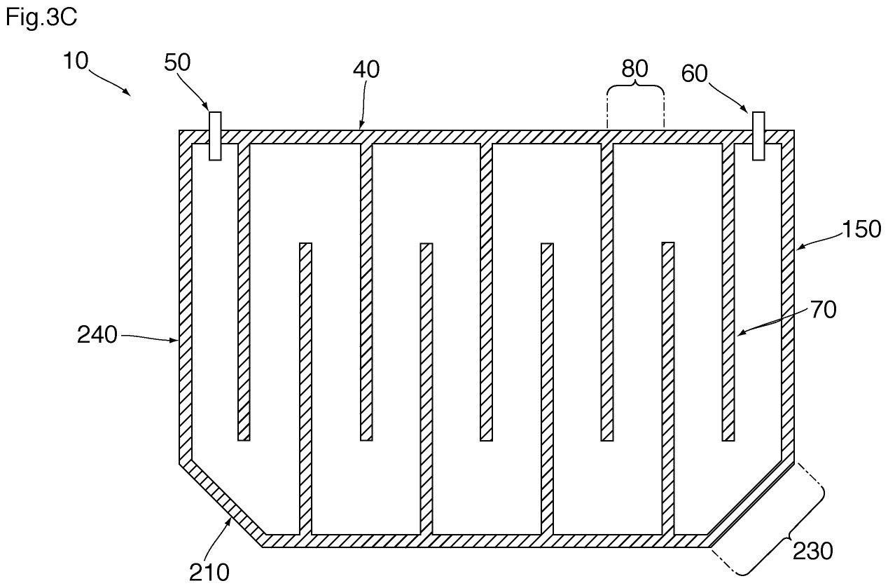

Referring now to FIGS. 3A and 3B, in the examples shown, which is shown sized and shaped suitably for a kidney, the fillable chamber 10 includes 7 retention members 70 between the first layer, optionally the upper layer 20 and the second layer, optionally the lower layer 30 creating a series of spaced veins 80 creating a continuous channel 90 in a solenoid pattern. In FIG. 3C, in the example show, which is shown sized and shaped suitably for a kidney, the fillable chamber 10 includes 9 retention members 70 between the first layer, optionally the upper layer 20 and the second layer, optionally the lower layer 30 creating a series of spaced veins 80 creating a continuous channel 90 in a solenoid pattern. As used herein, the term “solenoid pattern” refers to the configuration of the fillable chamber 10, wherein the retention member 70 commences from a longitudinal edge 40 and extends toward an opposite side of the fillable chamber 10, abutting a turning portion 230 in the fillable chamber 10 creating the extending portion 220 of the continuous channel 90. The commencing point of each retention member 70 alternates from opposing longitudinal edges.

Still referring to FIGS. 3A and 3B, specifically, the fillable chamber 10 is shown rectangular in shape with cropped corners 210, wherein the retention members 70 are spaced by veins 80 of about 1.5 cm wide. Each retention member 70 may be about 5 cm in length each extending portion 220 of the continuous channel 90 may be about 5 cm, and each of the turning portions 230 of the continuous channel 90 may be about 3 cm long and about 5 cm wide. Referring to FIG. 3C each retention member 70 is about 6 cm in length each extending portion 220 of the continuous channel 90 is about 6 cm and each of the turning portions 230 of the continuous channel 90 is about 2 cm long. Other lengths can also be used. In the example shown, the inlet 50 is positioned in an end portion 120 of the longitudinal edge 40 of the fillable chamber 10 between the first lateral edge 240 of the fillable chamber 10 and the retention member 70 and the outlet 60 is between the second lateral edge 150 of the fillable channel 10 and the last retention member 70. In other examples, the dimensions of the fillable chamber 10 are such to allow contact with a substantial portion of an organ.

In alternative examples, the fillable chamber 10 may include at least 3, at least 4, at least 5, at least 6, at least 7, at least 8, at least 9 or at least 10 retention members 70, depending for example on the size of the fillable chamber. In alternative examples, the fillable chamber 10 may include at least 10 retention members 70. In alternative examples, the fillable chamber 10 may be any geometric shape. In other examples, the one or more fillable chambers may be a variety of geometric shapes optionally adapted for a particular organ. In some examples, the one or more fillable chambers may be shaped in about the configuration of a triangle, rectangle, oval, trapezoid, hexagon, square or parallelogram. In alternative examples, the fillable chamber 10 may or may not include the cropped corners 210. In alternative examples, each retention member 70 may be separated by a vein 80 about 1.5 cm wide (between retention members 70 extending from opposing sides of the fillable chamber 10) and about 3 cm wide (between retention members 70 extending from the same side of the fillable chamber 10). In alternative examples, each of the turning portions 230 of the continuous channel 90 is dimensioned to be wider than the width of the extending portion 220 of the continuous channel 90, for example approximately or at least double the width of each extending portion 220 of the continuous channel 90. In alternative examples, the extending portion 220 of the continuous channel 90 may be the same length as the one or more retention members 70.

In other examples, the one or more fillable chambers (e.g. the plurality) are shaped in a variety of geometric shapes suitably adapted to a variety of organs. In some examples, the fillable chamber is a polygon approximating the surface of an organ. In alternative examples, the fillable chambers 10 may be sized and/or shaped suitably for a heart, lung, liver or pancreas. In some examples, the one or more fillable chambers may be shaped in the configuration of a triangle, rectangle, oval, elongated oval (oblong), circle, trapezoid, square, or any other polygon. In alternative examples, the temperature control, optionally cooling, apparatus 1 may comprise more than one fillable chamber 10 having differing geometric shapes. In some examples, two or more fillable chambers 10, optionally having one or more geometric shapes, may be used in a modular fashion, optionally wherein the two or more fillable chambers 10 are connected using for example one or more clips, snaps, adhesives, or fasteners 100. In alternative examples, the fasteners 100 may be Velcro™ or Steri-Strips™, or other hook and loop fasteners. In alternative examples the fasteners 100 may be snaps, buttons, buckles or other fasteners similar to fasteners 100. In alternative examples, the two or more fillable chambers 10 are welded or glued together or otherwise adfixed.

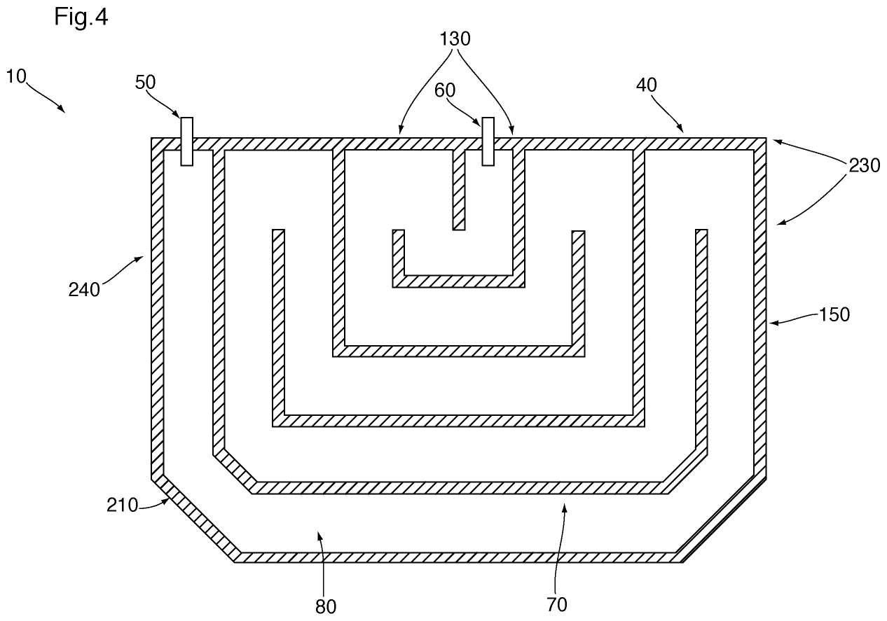

Referring now to FIG. 4, in the example shown, the fillable chamber 10 includes 5 retention members 70 between the first layer, optionally the upper layer 20 and the second layer, optionally the lower layer 30 creating a series of spaced veins 80 creating a continuous channel 90 in a U-shape pattern, wherein the each of the retention members 70 forms a U shorter than the one below it, but longer than the one above it. Specifically, the fillable chamber 10 may be rectangular in shape with cropped corners 210 wherein the retention members 70 are about 1 cm apart, each of the turning portions 230 of the continuous channel 90 being about 2 cm wide. In the example shown, the inlet 50 is in the end portion 120 of the longitudinal edge 40 of the fillable chamber 10 between the first lateral edge 240 of the fillable chamber 10 and the retention member 70; and the outlet 60 is between two retention members 70 in a middle portion 130 of the fillable chamber 10. In some examples, no portion of the continuous channel 90 may be narrower than other portions of the continuous channel 90, optionally 1 cm.

In alternative examples, the fillable chamber 10 may include at least 3 retention members 70. In alternative examples, the fillable chamber 10 may include at least 10 retention members 70. In alternative examples, the fillable chamber 10 may be any geometric shape. In other examples, the one or more fillable chambers may be any geometric shape suitably adapted to an organ. In some examples, the one or more fillable chambers may be shaped in the configuration of a triangle, rectangle, oval, circle, trapezoid, or square or other polygon. In alternative examples, the fillable chamber 10 may or may not include cropped corners 210 (FIG. 3C). In alternative examples, each retention member 70 may be between about 1 cm and about 2 cm apart. In alternative examples, each of the turning portions 230 of the continuous channel 90 may be at least double the width of each extending portion 220 of the continuous channel 90. In alternative examples, the extending portion 220 of the continuous channel 90 may be the same length as the one or more retention members 70.

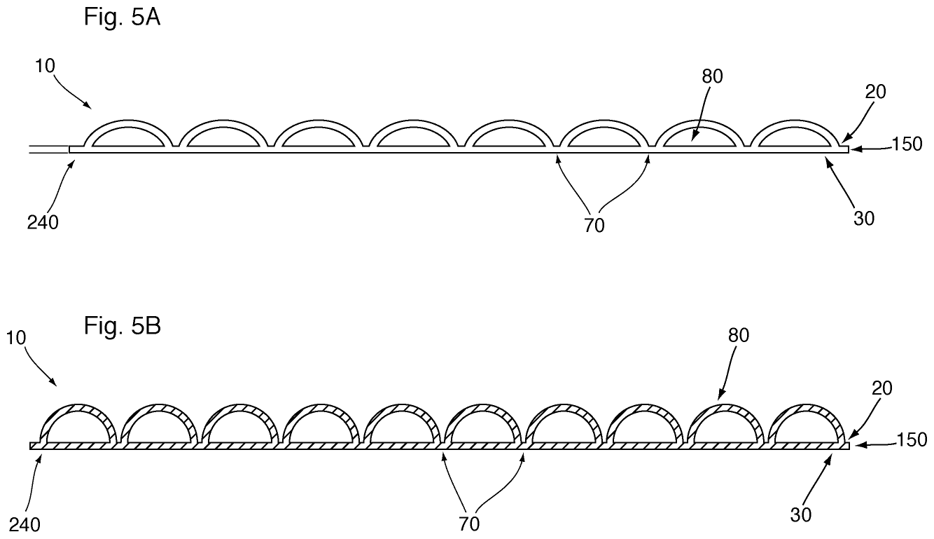

Referring now to FIG. 5A, in the example shown includes the fillable chamber 10 including 7 retention members 70 between the first layer, optionally the upper layer 20 and the second layer, optionally the lower layer 30 creating a series of spaced veins 80, wherein the combined thickness of the first layer, optionally the upper layer 20 and the second layer, optionally the lower layer 30 are about 0.1 cm to about 0.3 cm or less, and the spaced veins 80 are about 0.3 cm in height. In FIG. 5B, in the example shown the fillable chamber 10 includes 9 retention members 70 between the first layer, optionally the upper layer 20 and second layer, optionally the lower layer 30 creating a series of spaced veins 80. The spaced veins 80 can be about 0.3 cm or 0.4 cm in height. The retention members 70 can be sealed junctions where the sealed width may be less than the total width of the first layer, optionally the upper and second layers, optionally the lower layers 20, 30. In alternative examples, the veins may between about 0.1 cm to 0.4 cm in height, preferably 0.2 cm or 0.3 cm, and between about 0.9 cm to about 1.1 cm in width, preferably, about 1 cm in width. The retention members can for example be a bonding agent that bonds the first layer, optionally the upper and second layer, optionally the lower layers to create the retention member. In some examples, the retention members 70 are sealed using glue or other adhesive or bonding agent. FIG. 5B shows an embodiment where the spaced veins have a height of greater than 0.3 cm.

Referring now to FIGS. 6A and 6B, the example shown depicts circulation tubing 160 and a circulation fluid receptacle 140. In some examples, the circulation tubing 160 may be releasably connected to a circulation fluid source and releasably connected to the inlet 50 of a fillable chamber 10. In some examples the circulation fluid source may be a tap or faucet or circulation fluid receptacle 140. In some examples, the circulation fluid receptacle 140 may be a bag or other container suitable for carrying a reservoir of liquid. In some examples, the circulation fluid receptacle 140 may be or may be similar to a feeding bag or IV bag, optionally gas sterilized. In some examples, the circulation fluid receptacle 140 may be connected to a pump. In alternative examples, the circulation fluid receptacle 140 may be hung at least about 80 cm above the first and second fillable chambers 10.

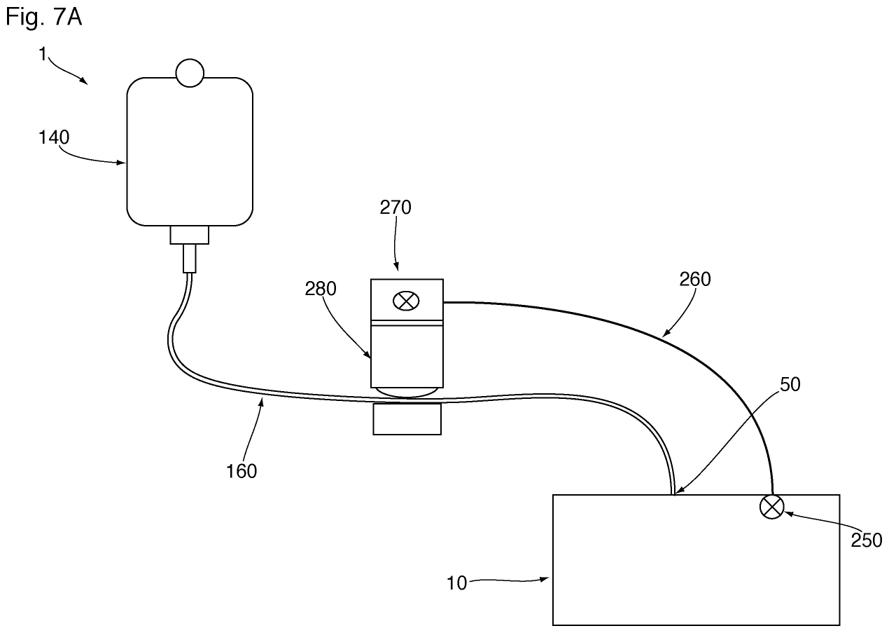

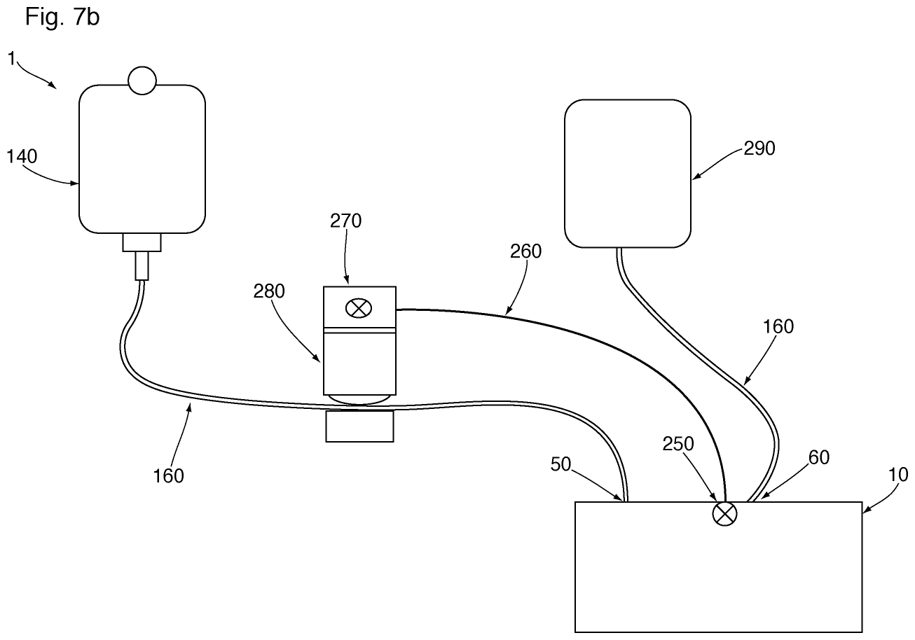

Referring now to FIG. 7A and FIG. 7B, the examples shown depict a temperature control, optionally cooling, apparatus 1 comprising one or more fillable chamber(s) 10, connected to a circulation fluid receptacle 140 by circulation tubing 160 connected to an inlet 50 optionally positioned at the periphery (FIG. 7B) or the center (FIG. 7A) of one of the one or more fillable chambers 10, further including a temperature sensor 250 capable of measuring organ surface temperature and/or transmitting organ surface temperature information for example by a transmitting wire 260 or wirelessly, to a receiver 270 operatively connected to a flow valve 280 on the circulation tubing 160 connecting the circulation fluid receptacle 140 and one or more fillable chamber(s) 10, wherein the flow valve 280 may be configured to alter or maintain the flow of the circulation fluid based on the information received from the temperature sensor 250 through the receiver 270. The flow valve 280 may be configured to effect one or more desired flow rates. In some examples, the flow valve 280 may have 3 settings to affect a slow, medium, or high rate of flow of circulation fluid from the circulation fluid receptacle 140, through the circulation tubing 160, to the one or more fillable chamber(s) 10. In other examples, the flow valve 280 may have 2 settings, one to wholly obstruct circulation fluid flow and/or one to allow unobstructed circulation fluid flow. In other embodiments, the flow valve 280 may be configured to permit any desired level of circulation fluid flow. In some examples, the flow valve 280 may be a pinch valve. In other examples, the flow valve 280 may be any valve capable of wholly or partially obstructing the flow of circulation fluid through the circulation tubing 160. The temperature sensor 250 can be configured to transmit information indicating the temperature of an organ or whether the organ surface temperature is within a selected range for example, a target range of 3 to 5° C., and for example be coupled with an alarm should the temperature rise above or below the selected range. In some examples, the temperature sensor 250 can be a temperature probe. In some examples, the temperature information can be transmitted to receiver 270 wirelessly. Other thermostatic controls can also be used to control the circulation fluid flowrate based on the organ surface temperature being cooled. In alternative examples, the receiver 270 may be operatively connected to a pump that alters or maintains the flowrate of the circulation fluid based on the information received from the temperature sensor 250.

The temperature sensor 250 may be placed on the outer surface of one of the layers, optionally of the second layer, optionally the lower layer 30 of one of the fillable chambers 10, optionally in the center of the fillable chamber 10 (FIG. 7B) and/or proximal to the outlet 60 (FIG. 7A) for example to control the maximum temperature of the exiting circulation fluid, optionally below about 4 to about 5 degrees Celsius, optionally between about 3 to about 5 degrees Celsius.

Referring now to FIG. 8A and FIG. 8B, the examples shown depict the temperature control, optionally cooling, apparatus 1 comprising a suspension member 310 that is distinct from the fasteners 100. As shown therein the temperature control, optionally cooling, apparatus comprises a first and second fillable chamber 10 and a suspension member 310 connected to each of the fillable chambers 10, wherein the suspension member 310 extends from a middle portion of the longitudinal edge 40 of each of the first and second fillable chambers 10. The temperature control, optionally cooling, apparatus 1 also comprises a first and a second fastener 100, wherein each of the fasteners 100 is attached to the first and second fillable chambers 10. The fasteners 100 may be secured in place using one or more securing members 300, wherein each of the securing members 300 are attached to at least one fillable chamber 10 creating a recess or beltloop in which the fasteners 100 are received; and a vessel holder 320. The securing members 300 are typically proximal to the edge opposite the elongated edge, optionally the longitudinal edge 40. The securing members 300 can be fixed to the fillable chamber 10 in a substantially aligned and/or parallel manner thereby creating a guide for cutting the fasteners, for example once an organ has been transplanted and/or the temperature control, optionally cooling, apparatus 1 is no longer needed. The securing members 300 in addition to securing the fasteners 100 act as cutting guides. Upon cutting, fillable chambers 10 are no longer fastened together and the temperature control, optionally cooling, apparatus 1 can be removed by first removing the fillable chamber 10 most accessible. The cut fastener 100 slides out of the securing members 300 like a belt pulled through a belt loop. The fasteners 100 can also be cut elsewhere.

In some examples, there may be one or a plurality of fasteners 100. In some examples, each of the fasteners 100 may be attached to one or more of the fillable chambers 10 comprised in the temperature control, optionally cooling, apparatus 1, but not all. In other examples, each of the fasteners 100 is attached to all the fillable chambers 10 comprised in the temperature control, optionally cooling, apparatus 1. In some examples, the fasteners 100 may be releasably attached to the first and second fillable chambers 10. In other examples, the fasteners 100 may be attached to the first and second fillable chambers 10 using an adhesive, for example, glue or other adhesive or bonding agent. In other examples, the fasteners 100 may be welded to the one or more fillable chambers 10.

In some examples, at least one suspension member 310 may be connected to or extend from at least one fill able chamber 10. In other examples, a first suspension member 310 may be connected to a first fillable chamber 10 and a second suspension member 310 may be connected to a second fillable chamber 10. In other examples, a plurality of suspension members 310 may be connected to a plurality of fillable chambers 10. In some examples, the at least one suspension member 310 may be releasably connected to the at least one fillable chamber 10. In some examples, the at least one suspension member 310 may be connected to the at the least one fillable chamber 10 using an adhesive, for example, glue or other adhesive or bonding agent. In other examples, the at least one suspension member 310 may be welded to the at least one fillable chamber 10.

In some examples, the at least one securing member 300 may be attached to at least one fillable chamber 10. In some examples, the at least one securing member 300 may be releasably attached to the at least one fillable chamber 10. In some examples, the at least one securing member 300 may be attached to the at least one fillable chamber 10 using an adhesive. In some examples, the at least one securing member 300 may be welded to the at least one fillable chamber 10.

In some examples, the at least one vessel holder 320 may be attached to at least one fillable chamber 10. In some examples, the at least one vessel holder 320 may be releasably attached to the at least one fillable chamber 10. In some examples, the at least one vessel holder 320 may be attached to the at least one fillable chamber 10 using an adhesive. In some examples, the at least one vessel holder 320 may be welded to the at least one fillable chamber 10. In some examples, the at least one vessel holder 320 may be made from silicone or vinyl or any other suitable material.

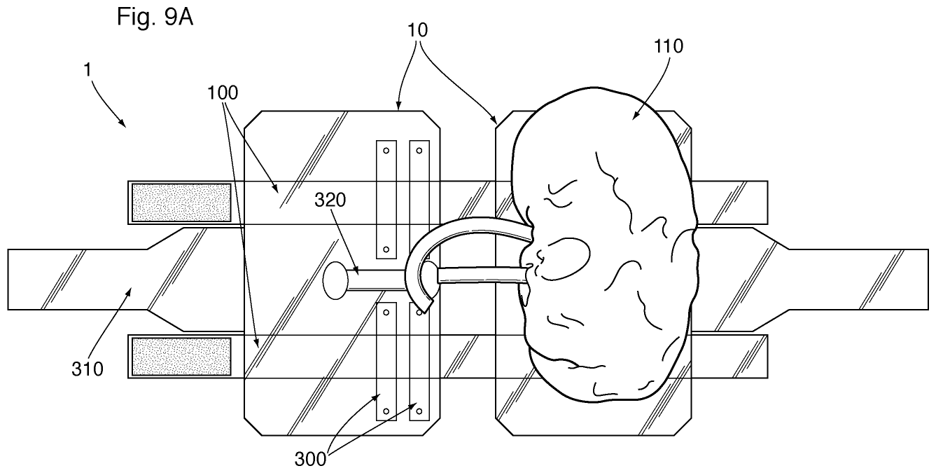

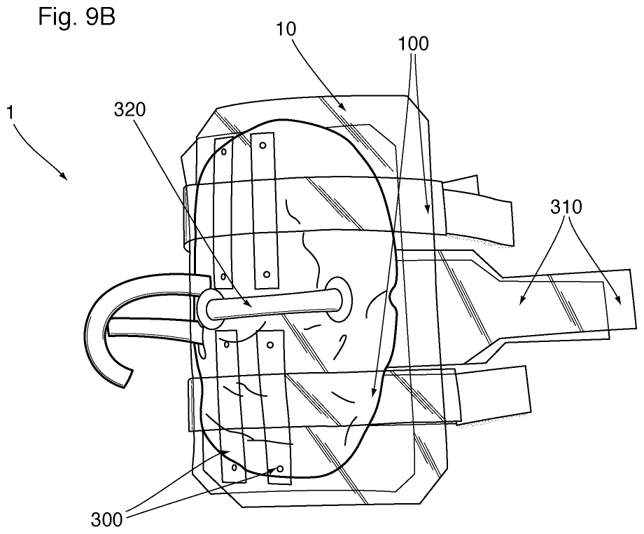

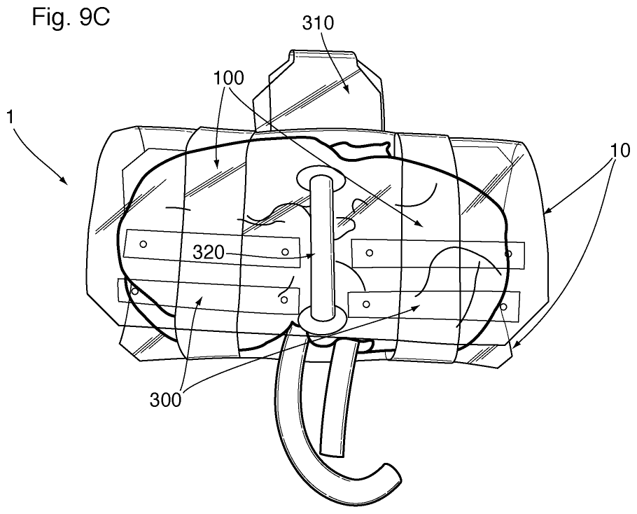

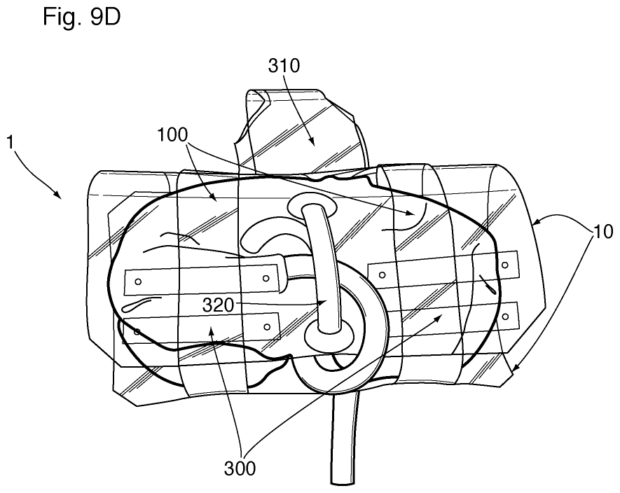

Referring to FIGS. 9A, 9B, 9C, and 9D, the examples shown depict a model of fillable temperature control, optionally cooling, apparatus 1 of FIGS. 8A and 8B adapted to contact a model organ.

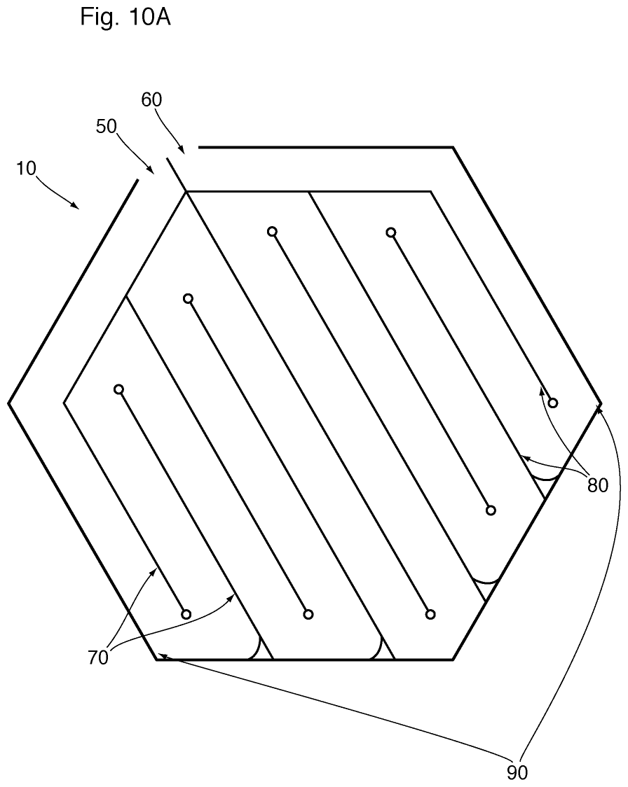

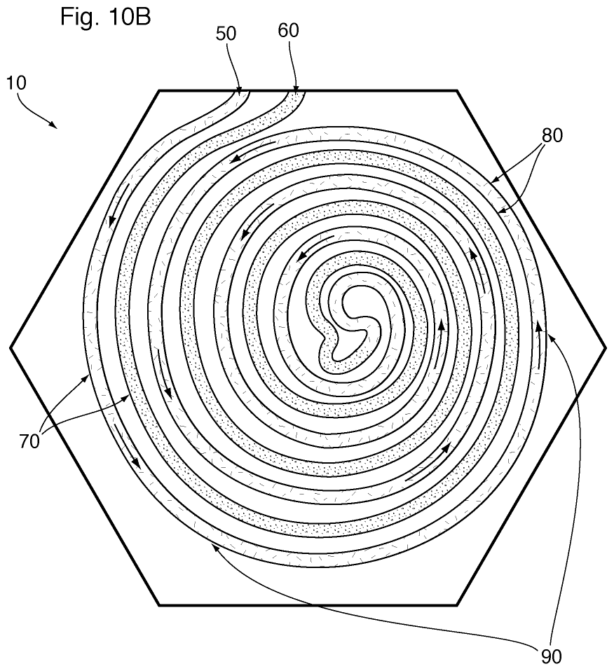

Referring now to FIG. 10A, in the example shown, the fillable chamber 10 is hexagonal in shape and includes 9 retention members 70 creating a series of spaced veins 80 creating a continuous channel 90. In the example shown, the inlet 50 is adjacent to the outlet 60 of the fillable chamber 10. In alternative examples, the inlet 50 and outlet 60 are distanced apart, for example on the same edge or on opposing sides of the fillable chamber 10. In the example shown, the retention members 70 are generally straight or uncurved. In alternate embodiments, the retention members 70 can include a curve as shown in FIG. 10A. In alternative examples, the fillable chamber 10 may include at least one retention member 70, optionally 2, 3, 4, 5, 6, 7, 8, 9, 10, 11, 12, 13 14, 15, 16, 17, 18, 19, or 20 retention members 70. In alternative examples, the fillable chamber 10 may include at least 9 retention members 70. In the example shown, 4 of the spaced veins 80 include a curve and 6 spaced veins 80 do not, e.g. are uncurved. In other embodiments, all of the spaced veins 80 are curved or all of the spaced veins 80 are uncurved. In some embodiments, half of the spaced veins 80 are curved and/or half of the spaced veins 80 are uncurved.

Referring now to FIG. 10B, in the example shown, the fillable chamber 10 is hexagonal in shape and includes 2 retention members 70 creating a continuous series of curved spaced veins 80 approximating concentric circles and creating a curved continuous channel 90. In the example shown, for example when used for cooling, the lined pattern represents cold circulation fluid, while the dotted pattern represents the circulation fluid once it is no longer cold. In the example shown, for example when used for warming, the lined pattern represents warm circulation fluid, while the dotted pattern represents the circulation fluid once it is no longer warm. In the example shown, the arrows depict the flow path of the circulation fluid. In the example shown, the inlet 50 adjacent to the outlet 60 of the fillable chamber 10. In alternative examples, the inlet 50 and outlet 60 are on opposing sides of the fillable chamber 10. In the example shown, the retention members 70 are curved. In alternative examples, the fillable chamber 10 may include at least retention members 70. In one example, the fillable chamber 10 of FIG. 10A or FIG. 10B can be made by affixing a first end of the retention members 70 to a first layer and affixing a second layer to a second end of the retention members, optionally wherein the affixing may be done with a bonding agent. In other examples, the fillable chamber 10 of FIG. 10A or FIG. 10B can be made using a heated press, for example a linear or curved heated press. In other examples, the fillable chamber 10 of FIG. 10A or FIG. 10B can be made using one or more of the methods described in Example 3.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

### EXAMPLES

### Example 1: Models and Protocol Tests in Kidneys with a Pump

The organ cooling device has a thin, non-bulky design that does not get in the way of the transplant surgery and allows visualization of the vessels trying to be connected. The temperature can be maintained at 3-5° C. The configuration of the device, which is very unique, ensures that fluid is always on the move (better heat transfer and more uniform).

Models and protocol tests have shown that we can maintain a temperature of 4° C. for over one hour with a flow rate of 900 cc/hr and a thickness of 2 mm for each side of the organ temperature control or cooling device. The device contains saline, a physiological fluid, and not alcohol or water, which could injure the kidney if they ever leaked out. Practical considerations, such as access to the vessels and a means of holding the kidney so it does not fall out of the device, have been addressed.

Saline could be infused into the two fillable chambers using a regular intravenous pump found in all operating rooms. It was shown that a surface temperature of 4° C. could be maintained with a flow rate of 900 cc/hr which all intravenous pumps can achieve.

The kidney model was placed between two solenoids, with the direction of flow in opposite directions, the anterior one proceeding from left to right, whereas the posterior one proceeding from right to left. An ice water bath was used to provide non-sterile water at a temperature of 2 to 2.5° C. The cooled water was delivered to the device tubing via 200 mL syringe using an injector pump. Both the control and cooled “kidney” were cooled to a starting temperature of 4.5°. The device was flushed with cooling solution for about 100 mL to cool the device as well. Temperature was measured using a small thermocouple placed on the surface of the “kidney” as well as an infrared no touch thermometer. We started with a rate of about 900 mL per hour but found that reducing the flow to 600 mL per hour maintained the ideal temperature. At the end of 35 minutes, several temperatures were taken from different parts of the kidney. The measurements varied between 3.2 and 4.5° C. The control “kidney” at a temperature of 17° C. at the end of the 35 minutes.

### Example 2: Model and Protocol Test in Kidneys with No Pump

A feeding tube bag was used to infuse circulation fluid into the device. It has a large opening at the top to allow ice to be added to 2-4° C. ice water that flows through the device. This device worked well with gravity alone and the organ cooling device. The ice floated to the top of the bag. The thin design allowed for better contact with the kidney. The tubing from the bag is the same diameter (3 mm) as standard iv tubing.

The starting temperature was 4.3° C. for both kidneys. The test ran for 40 minutes and involved gravity feed of ice water via tube feed bag. The bag was hung at about 80 cm above the kidney (hook on bookshelf above the workbench). 500 cc was infused in 40 minutes (that would be 750 cc/hr). After 40 minutes, the temperature of the kidney with organ cooling device attached was 3.4° C. to 4.6° C. Control kidney temperature was 26° C. at 40 minutes. No pump was used. The thin design increased the resistance of the system and the contact with the kidney.

The kidney model was placed between two solenoids, with the direction of flow in opposite directions, the anterior one proceeding from left to right, whereas the posterior one proceeding from right to left. A 1-liter feeding tube bag, which has the benefit of a large opening at the top allowed us to fill it with ice cubes and water. The ice cubes floated to the surface, and all that flowed through the tubing by gravity was the cooled water which measured approximately 2° C.

Both the control and cooled “kidney” were cooled to a starting temperature of 4.3° C. The device was flushed with cooling solution for about 100 mL to cool the device as well. Temperature was measured using a small thermocouple placed on the surface of the “kidney” as well as an infrared no touch thermometer.

Using the roller crimp valve that came attached to the tubing for the feed tube bag, the rate was adjusted to about 750 mL per hour. The test ran for 40 minutes. The final temperature of the cooled kidney was 3.4 to 4.6° C., whereas the temperature of the control kidney was 26° C.

### Example 3: Method of making the device

One method of making the organ temperature control device described herein (FIGS. 1A, 1B and 1C) was by using laminating sheets and a vacuum sealer.

Another method of making the organ temperature control device described herein is with transparent vinyl and a heat sealer (FIG. 3A). A heat sealer was used to create the fillable chambers out of a sheet of vinyl, and to create the retention members 70 in the pattern shown in FIG. 3A. Its transparency is useful for visualization of the kidney.

A small spacer was used to allow for the anterior layer of polyvinyl to bubble to a reproducible and uniform channel size. For example, a 4-inch bolt having a diameter of about 3 mm or a copper wire was used. The diameter of the wire is slightly less than 1 mm. Any other spacer can also be used

A plastic sealer and two sheets of polyvinyl chloride were used to introduce retention members 70 or veins in the same direction. The edges of the chambers 10 are sealed or otherwise joined leaving an inlet 50 and/or an outlet 60. The tubing is placed through the inlet 50 and/or outlet 60 to create a watertight junction. The tubing can be affixed. For example, the tube in some models was held place using gorilla glue. Various fixatives and adhesives can be suitably used.

The chamber 10 can be produced in a three-layer manufacturing method. For example, the assembly would comprise placement of a first sheet laid on a surface, placement of retention members 70 in a desired pattern on the first sheet and a second sheet that is placed on the retention members 70. The retention members 70 be anything that bonds to the first sheet and second sheet, for example the retention members 70 may be a bonding agent. For example, the first and/or second sheets can be clear flat vinyl, PVC or silicone sheets with a 3D printed precut pattern of veins sandwiched between the top and bottom sheet. The precut pattern retention member 70 layer could be as little as 0.5 or 1 mm thick. This would have the advantage of allowing just about any solenoid pattern to be produced.

The chambers are then connected optionally with clips 200 and/or one or more fasteners 100 and/or the various connector tubes and/or other components are added.

In some embodiments, one or more of the various connector tubes and/or other components are fused to the chambers 10 at the time of manufacturing. For example, a portion of flat tubing can be fused to the chambers 10 at the time of manufacturing. The fused flat tubing can have a connector such as a Luer connector, on the non-fused end, allowing it to be compatible for connection with other components, for example several types of medical tubing.

### Example 4: Model Comprising Thermostatic Control

Organ temperature can be maintained at or between a desired range, e.g. about or between 3° C. and 4° C. using a sensor that measures the temperature of the organ, transmits the temperature information to a receiver controlling a valve, the receiver is adapted to cause the valve to alter or maintain the flow rate of circulation fluid rom a circulation fluid receptacle to the fillable chambers contacting the organ (FIG. 7).

1. A fillable chamber or a plurality of fillable chambers each having
a first layer,
a second layer,
an inlet and an outlet, and
retention members between the first layer and second layer in a series of spaced veins creating a continuous channel extending from the inlet to the outlet.

2. (canceled)

3. The fillable chamber(s) of claim 1, wherein the plurality of fillable chambers comprises 3 or more, 4 or more or 5 or more fillable chambers; wherein the fillable chambers are of varying shape, optionally wherein one or more are about trapezoidal, arc comprisingcomprise a tapered end, are about triangular, are about rectangular, are about oval, are about hexagonal, or are about circular, and/or wherein the fillable chamber are of varying size.

4. (canceled)

5. The fillable chamber(s) of claim 1, wherein one or more of the fillable chambers are adapted to contact a kidney surface, a liver surface, a heart surface, a lung surface, a pancreas surface, or a digit, a hand, a face or tissue flap surface and/or adapted to be modular; and/or wherein one or more of the fillable chambers comprise a suspension member, one or more securing members and/or a vessel holder.

6-10. (canceled)

11. A temperature control, optionally cooling, apparatus comprising:
the plurality of fillable chambers of claim 1;
one or more fastener(s) for securing the plurality of fillable chambers, wherein the plurality of fillable chambers are adapted to contact a surface of an organ; and
optionally wherein each fillable chamber comprises a suspension member, one or more securing members, and/or a vessel holder.

12. The apparatus of claim 11, wherein the apparatus comprises:
a first fillable chamber having
a first layer,
a second layer,
an elongated edge, preferably a longitudinal edge,
an inlet and an outlet in the elongated edge, preferably longitudinal edge, and
retention members between the first layer and second layer in a series of spaced veins creating a continuous channel extending from the inlet to the outlet;
a second fillable chamber having
a first layer,
a second layer,
an elongated edge, optionally a longitudinal edge,
an inlet and an outlet in the elongated edge, preferably longitudinal edge,
retention members between the first layer and second layer in a series of spaced veins creating a continuous channel extending from the inlet to the outlet; and
optionally one or more fastener(s) for securing the first chamber and the second chamber, wherein the first chamber and the second chamber are adapted to contact a surface of an organ; and
optionally wherein each fillable chamber comprises a suspension member, one or more securing members and/or a vessel holder.

13. The apparatus of claim 12, wherein the first and second fillable chambers are constructed of a flexible, resilient material, optionally a transparent or translucent flexible, resilient material, optionally vinyl or silicone; wherein the continuous channel is configured in a solenoid pattern or a circular pattern or a U-shape pattern; and/or wherein the organ is a kidney, lung, pancreas, heart or liver.

14-15. (canceled)

16. The apparatus of claim 12, wherein the elongated edge is a convex edge; wherein each of the first and second fillable chambers comprise at least 2, at least 5, at least 7, at least 9 or more retention members; wherein the inlet and outlet further comprise a fitting, optionally a valve or luer lock attachment and/or the inlet and outlet are in opposite ends of the elongated edge, preferably longitudinal edge of each of the first and second fillable chambers.

17-19. (canceled)

20. The apparatus of claim 12, wherein
the inlet of the first fillable chamber is configured to be aligned with the inlet of the second fillable chamber and the outlet of the first fillable chamber is configured to be aligned with the outlet of the second fillable chamber when in use or wherein the inlet of the first fillable chamber is configured to be aligned with the outlet of the second fillable chamber and the outlet of the first fillable chamber is configured to be aligned with the inlet of the second fillable chamber when in use.

21-22. (canceled)

23. The apparatus of claim 12, wherein
the inlet is positioned towards an end portion of each of the first and second fillable chambers and the outlet positioned in a middle portion of each of the first and second fillable chambers.

24. (canceled)

25. The apparatus of claim 12, further comprising one or more circulation tubings wherein the inlet and outlet are releasably coupled to the circulation tubing and/or a temperature sensor on the surface of the organ to measure its temperature and transmit such temperature information to a flow valve operatively connected to the one or more circulation tubings or a pump operatively connected to a circulation fluid receptacle.

26-27. (canceled)

28. A method of cooling, warming, and/or maintaining the temperature of an organ prior to or during a transplant procedure, the method comprising
contacting the organ with the apparatus of claim 11,
securing the apparatus to the organ;
attaching the inlets of the first and second fillable chambers of the apparatus to a circulation fluid source; and
infusing circulation fluid into the inlet, through the continuous channel, and out the outlet of each of the first and second fillable chambers of the apparatus, wherein the temperature of the circulation fluid is selected to cool, warm or maintain the temperature of the organ.

29. The method of claim 28, wherein the first fillable chamber contacts a substantial portion of a first surface of the organ, optionally at least about 70%, optionally 80%, preferably 90% of the first surface of the organ and the second fillable chamber contacts a substantial portion of a second surface of the organ, optionally at least about 70%, optionally 80%, preferably 90%, of the second surface of the organ.

30. The method of claim 28, wherein the apparatus is secured to the organ using one or more fastener(s) for securing the first and second fillable chambers to the organ; wherein the inlets of each of the first and second fillable chambers are connected to a fitting, optionally a valve or luer lock attachment; and/or wherein the tubing is attached to the circulation fluid source, optionally a circulation fluid receptacle, optionally a bag, comprising a circulation fluid, optionally wherein the circulation fluid is saline or water.

31-36. (canceled)

37. The method of claim 28, wherein the infusing is suitable to achieve a flowrate of between about 600 cc/hour to about 1000 cc/hour, optionally about 750 cc/hour, optionally wherein the flowrate is achieved using a pump or gravity, and/or wherein the flowrate is altered or maintained by the flow valve in response to information received from the temperature sensor on the surface of the organ.

38-40. (canceled)

41. The method of claim 28, wherein the infusing is suitable to reduce the organ surface temperature to or maintain the organ surface temperature at a temperature of hypothermic metabolic arrest of the organ, optionally between about 3° C. and about 6° C., optionally between about 3.4° C. and about 4.6° C. and/or to reduce the organ surface temperature gradually.

42-43. (canceled)

44. The method of claim 28, wherein the organ surface temperature is maintained for, or the organ is cooled for, at least or about 40 minutes.

45-49. (canceled)

50. The method of claim 28, wherein the infusing is suitable to increase the organ surface temperature to or to maintain the organ surface temperature between about 10° C. and about 38° C., between about 15° C. and about 38° C., between about 20° C. and about 38° C., between about 25° C. and about 38 ° C., or between about 30° C. and about 38° C. and/or the infusing is suitable to increase the organ surface temperature gradually: and/or wherein the organ is a kidney, lung, pancreas, heart, liver or tissue.

51-53. (canceled)

54. A method of making the apparatus of claim 11, comprising contacting a panel of flexible, resilient material, optionally vinyl or silicone, with a heating device, optionally a sealer, to fuse areas of the panel, thereby creating the series of spaced veins and the continuous channel.

55. The method of claim 54, wherein the continuous channel is configured in a solenoid pattern or a U-shape pattern.

56-58. (canceled)

59. A kit comprising the apparatus of claim 11, or one or more the fillable chambers of the apparatus, and optionally one or more fastener(s) and/or side fasteners, a pump, circulation tubing, a circulation fluid receptacle, a temperature sensor, a receiver and flow valve, and/or a transmitting wire, optionally wherein one or more of the components are packaged in sterile condition.

60-68. (canceled)
